(19) United States
(12) Patent Application Publication
(10) Pub. No.: US 2014/0018609 A1
(43) Pub. Date: Jan. 16, 2014

(54) SLEEP ASSISTANCE ARTICLE AND RELATED METHODS OF USE

(71) Applicant: EDIA LLC, Minneapolis, MN (US)

(72) Inventors: James Howard, Birmingham, AL (US); Jeff Julkowski, Naples, FL (US)

(21) Appl. No.: 13/941,396

(22) Filed: Jul. 12, 2013

Related U.S. Application Data
(60) Provisional application No. 61/671,477, filed on Jul. 13, 2012.

(51) Int. Cl. A61M 21/02

(52) U.S. Cl. CPC A61M 21/02
USPC 600/28

(57) ABSTRACT

An article and related method to train a reflexive action, including helping a person fall to sleep. The article includes an electronic component configured to receive electrical signals, including electrical signals associated with the generation of sounds and/or motion, and a generator for emitting selectable sounds and/or causing motion when sufficient pressure is applied to the article. The article includes different types of pressure sensors. The method includes the steps of programming the processor to generate sounds of selectable type for selectable periods of time that encourage a person to carry out a desired behavior, such as keeping the person's head on a pillow.

### CROSS REFERENCE TO RELATED APPLICATION

The present invention is a nonprovisional and claims the priority benefit of U.S. provisional application Ser. No. 61/671,477, filed Jul. 13, 2012, entitled SLEEP ASSISTANCE PILLOW owned by a common assignee. The entire content of the priority application is incorporated herein by reference.

### BACKGROUND OF THE INVENTION

1. Field of the Invention

The present invention relates to systems and methods used to affect behaviors. More particularly, the present invention relates to articles that can be used for reflexive training to teach desired behaviors and to adjust undesired behaviors. Still more particularly, the present invention relates to articles that aid in the teaching or modification of the sleep characteristics of a child but is not limited thereto. The invention includes a substrate and associated electronic component that help a child learn to fall asleep.

2. Description of the Prior Art

It has long been the nature of humans to learn and to teach that which has been learned. There are positive learned behaviors, negative learned behaviors and necessary behaviors that may not be considered either positive or negative. In general, it is a goal to encourage or develop positive behaviors and discourage or correct negative ones. It is believed that desired behavior is most favorably created when positive reinforcement is provided to the subject. The concept was arguably most famously documented by Pavlov.

Given this understanding of human nature and the related concept of reflex training, devices, system and other forms of technology have been developed to assist individuals train other individuals to behave in a desired manner. While the focus of the description of the present invention is directed to an article designed to help a child learn to fall asleep, the invention is not limited to that particular learned behavior. Instead, it is a mechanism that can be used to encourage a desired behavior based on certain conditions established through use of the article.

Pillows are widely used by people as a support for their heads when lying down and, particularly, when they lay down to sleep. There are some people, including children primarily but not limited to them, who have difficulty getting themselves to sleep. For example, when a child is put to bed and he or she seems to be unready to sleep, the child may resist sleep by not “settling” their head on the pillow and remaining still enough to fall asleep. That may happen eventually, but it can take much more time than is otherwise necessary for that to occur. It would be desirable have an article and/or method to encourage the person to keep his or her person still with his or her head in a sleep position.

Pillows have been developed that include components selected to generate sound. That is, they include electronics and speakers that play music or stories. They can be activated and deactivated by actuating an on/off switch or button. Other articles such as, toys, dolls and the like, include sound making equipment used to make music, stories and customized sounds. These pillows and other articles have such functionality but they are not configured to generate desired sounds in a manner that optimizes the conditions that encourage a person, such as a child, to remain in a position that is likely to lead to falling asleep as quickly as possible. What is needed is an article and related method of using the article to generate sounds in a way that encourages sleep.

In the course of the development of the present invention the inventors have observed that an article and related method of use that encourages a child to develop a positive approach to falling to sleep may be an effective reflexive training tool. Some studies appear to suggest that this type of approach may be of use in addressing concerns about the difficulties that arise when trying to get a child to fall to sleep. It is believed that future studies will support this view. In the meantime, it appears that what is needed is an article and related method that implement such capability. Beyond that and more generally, what is needed is an article and related method that can be used to assist in the reflexive training of individuals to achieve desired behaviors.

### SUMMARY OF THE INVENTION

It is an object of the present invention to provide an article and related method designed to aid in reflexive training. Further, it is an object of the invention to provide such an article and related method to help a person fall to sleep. The article includes a substrate, support or other type of retaining element to retain therein or thereon an electronic device that generates an audible or physical signal associated with an action undertaken, including the action of falling to sleep. The electronic component is configured to receive electrical signals, including electrical signals associated with the generation of sounds and/or motion, and a sound and/or motion generator for emitting selectable sounds or creating selectable movement. In addition, the electronic component includes a processor for processing instructions received from a source regarding sounds and/or motion to be generated and the details thereof, as well as to control operation of the sound and/or motion generator based on those instructions. The electronic component also includes memory, or at least a flash drive for receiving information and instructions. The article may optionally include an access port for receiving a memory card, flash card or other form of physical device configured to supply information and/or instructions to the processor. The electronics may be contained in a waterproof housing.

The article may be a pillow, a pillow accompaniment, a head rest, a crib mat, mat, bedding, a removable or a fixed car seat, a stuffed animal or any other thing that an individual to be trained would likely contact in association with the behavior to be modified. The article also includes a pressure sensing element that is coupled to the electronic component in a manner that activates or deactivates the sound transmitter and/or motion generator as a function of the pressure applied to or removed from the article. The article may include one or more covering components to cover the electronic component and the pressure sensing element with a way to access those components as desired. That is, the covering component may include one or more access ports for access to the interior thereof where the electronic component and the pressure sensing element are contained. For example, an access port may be used to access one or more batteries used to power the electronic component. The article, such as a pillow, may include foam, down, plastic or some other type of cushioning or fill. That optional cushioning or fill is placeable around the electronic component and the pressure sensing element within the one or more covering components. The article may include a body having an electronic component containment cavity and an access module conduit.

In an embodiment of the invention, the electronic component is coupled to an access module that extends through the containment component of the article. The access module may be used to contain replaceable batteries for powering the electronic component and/or a transceiver. It may also include one or more indicators, such as one or more lights and/or one or more sound ports for indicating whether the electronic component is on, receiving electrical signals and/or generating sound and/or motion. The access module may also include one or more elements for receiving and/or transmitting electrical signals, such as a radio card but not limited thereto.

The electronic component of the article includes a transceiver that may be physically connected to a source of electrical signals, such as with a USB cable, and/or wirelessly connected to such a source of electrical signals, such as through a Bluetooth transceiver, a wireless transceiver, a combination thereof or such other transceiver device generally available to transmit and receive electrical signals wirelessly. Wi-Fi connectivity may be used for the present invention. As noted, the transceiver may be located in the optional access module. Dependent upon the form of the transceiver, the article may be local to, or remote from, the source of electrical signals, wherein the electrical signals may include, but not be limited to, instructions for operation of the processor and the associated sound generator and/or motion generator.

The processor is programmed to generate or halt the generation of sound and/or motion as a function of the pressure applied to the pressure sensing element. The processor is also programmed to generate, with respect to sound generation, particular sounds, such as music, voices or other auditory noise, which sounds may be selected for generation, and/or it may be programmed to generate, with respect to motion generation, particular movements. The processor is further programmed to vary sounds/motion and generate sounds/motion for particular periods of time, which periods of time may be continuous, periodic or random. The programming of the processor may be updated or otherwise revised through wireless connectivity, wired connection to a source or other means of signal delivery. The memory of the electronic component may be updated in a similar manner.

The pressure sensing element, also referred to herein as the pressure sensor, is positioned within the article. When a body part, such as a head, leg or stomach, for example, makes contact with the exterior surface of the article, pressure is exerted on the article body and pressure is applied to the pressure sensor. Of course, other forms of pressure may be applied, not limited to a specific body part or parts. The pressure sensor is configured to convert that pressure into an electrical signal. Once that electrical signal, such as in the form of a current, reaches a certain level, it activates the processor to signal the sound/motion generator to generate sound/motion. It may also be configured to deactivate the processor based on that electrical signal generated by the pressure sensor.

The article can be used to carry out a method to help a person change or learn a behavior, including a child falling to sleep in a certain way, for example. The discussion of that method described herein will focus on a process for helping a young child to go to sleep; however, it may be used as a sleep or other reflexive training aid by any person. The method includes the step of programming the processor with instructions to generate selectable sounds/motion upon activation. That programming may be carried out before or after placing the electronic component within the article. The method includes the step of configuring the processor to generate the selectable sound/motion when pressure applied to the pressure sensor is sufficient to produce an electrical signal that activates the processor. The method also includes the step of selecting a pressure sensor configuration that generates the processor activating signal when the pressure applied to the pressure sensor is equivalent to the pressure applied by the person when settled or resting in a desired position on the article. For a child of age four to be trained to get himself or herself to sleep, for example, that pressure may be equivalent to the weight of an average child's head. That is, when the child's head is fully resting on the article, sound and/or motion is generator by the article, which may be a pillow in the case of a child to be trained to fall to sleep on an article recognized by the child to associate with sleep. It may be a pillow, a mat or a head rest, for example. When the person's head is off the article or not fully resting on it, the sound/motion is off. Alternatively, a sound may be generated informing the child of the status of the body position with respect to the article. The information may include an instruction for getting the child to return to the position desired to optimize a sleep condition. For example, the phrase “Lay your head down to hear the rest of the story” or similar type of phrase may be emitted.

An aspect of the method associated with the article is to educate the child to keep his/her head, etc., on the article in order to hear a desirable sound or feel a desirable motion. That condition provides a positive reinforcement of the desired behavior of falling to sleep. By the same token, movement of the head off the article results in a stoppage of the desired condition, thereby encouraging the child to change position to return to that condition.

The process for generating sound/motion and halting the generation of that sound/motion can be useful in the method to help a person learn or change a behavior, such as getting a child to fall to sleep in an effective way. In the example of a child, it is not uncommon for young children to fidget at bedtime. As a result, there can be a substantial amount of time between getting into bed and falling asleep. However, to the extent the process of falling to sleep can be accelerated by encouraging the child to keep his or her head still on a pillow, the article and method of the present invention provide the way to achieve that acceleration. In particular, the method of the present invention enables a user to choose one or more sounds that a child enjoys hearing and/or a form of motion the child enjoys hearing. The child may be part of the sound/motion selection process. The child learns that the sound may be heard and/or the motion felt when the processor is activated and that that occurs when the child fully rests his or her head on the pillow article.

An aspect of the method optionally includes the step of configuring the processor and the sound/motion generator to generate the sound/motion as soon as possible when contact is made with the article to encourage continuing contact with the article. Otherwise, the child also learns that when the head is lifted from the pillow or even moved about on the pillow, the sound/motion stops. The child quickly learns to keep the head still and on the pillow to hear the desired sound, which may be music, a story, a parent's voice or even white noise, but not limited thereto, and/or to feel desired movement. The stillness resulting from maintenance of the head on the pillow is more likely than not to lead to sleep more quickly than may otherwise occur. It can be seen that this may be used by people other than small children for a similar purpose and for other types of behavior learning or modification.

The article and related method of the present invention provide an advantage in helping a person learn or change a behavior, including the process for a young child to learn to fall to sleep. This and other advantages of the invention will become more apparent upon review of the following detailed description, the accompanying drawings and the appended claims.

### BRIEF DESCRIPTION OF THE DRAWINGS

FIG. 1 is a perspective top view of a pillow embodiment of the article of the present invention.

FIG. 2 is a perspective side view of the pillow article of the FIG. 1 embodiment of the present invention.

FIG. 3 is a cross sectional perspective view of the pillow article of the FIG. 1 embodiment of the present invention showing a pressure sensor and electronics housing component.

FIG. 4 is a top perspective view of four stages of the forming of the pillow article of the FIG. 1 embodiment of the present invention.

FIG. 5 is a perspective view of the electronics housing component of the FIG. 1 embodiment of the pillow article of the present invention.

FIG. 6 is a perspective bottom view of the fill component of the pillow article of the FIG. 1 embodiment of the present invention showing the electronic housing component retained in a cavity thereof and an access module with extension tube in an access module conduit thereof.

FIG. 7 is a perspective bottom view of a first cover of the pillow article of the FIG. 1 embodiment of the present invention showing a battery access port.

FIG. 8 is an exploded view of the pillow article of the FIG. 1 embodiment of the present invention showing a first embodiment of the pressure sensor.

FIG. 9 is a phantom view of the pillow article of the FIG. 1 embodiment of the present invention showing a second embodiment of the pressure sensor.

FIG. 10 is a cross sectional side view of a third embodiment of the pressure sensor.

FIG. 11 is a cross sectional side view of a fourth embodiment of the pressure sensor.

FIG. 12 is a cross sectional side view of a fifth embodiment of the pressure sensor.

### DETAILED DESCRIPTION OF THE PREFERRED EMBODIMENTS OF THE INVENTION

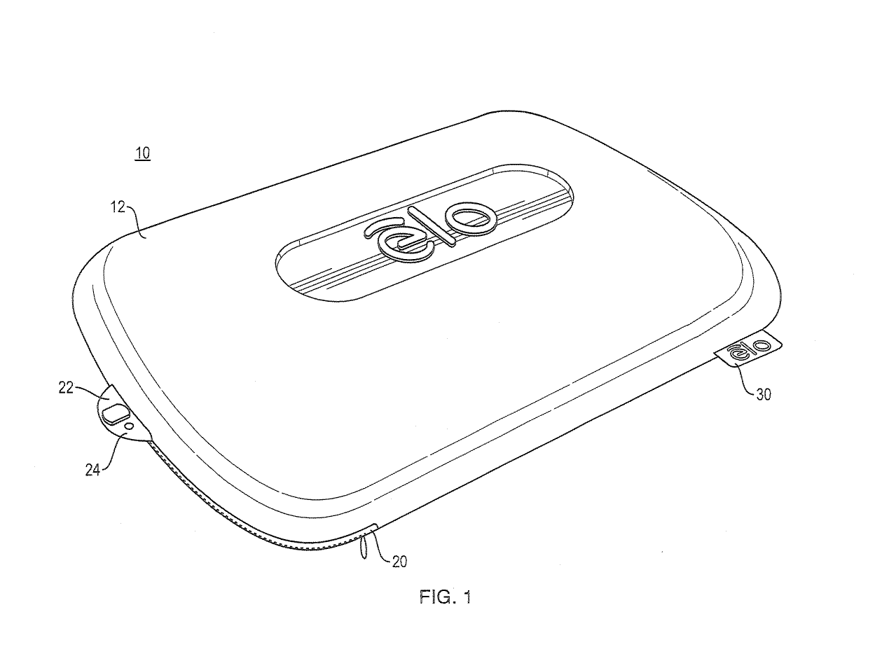

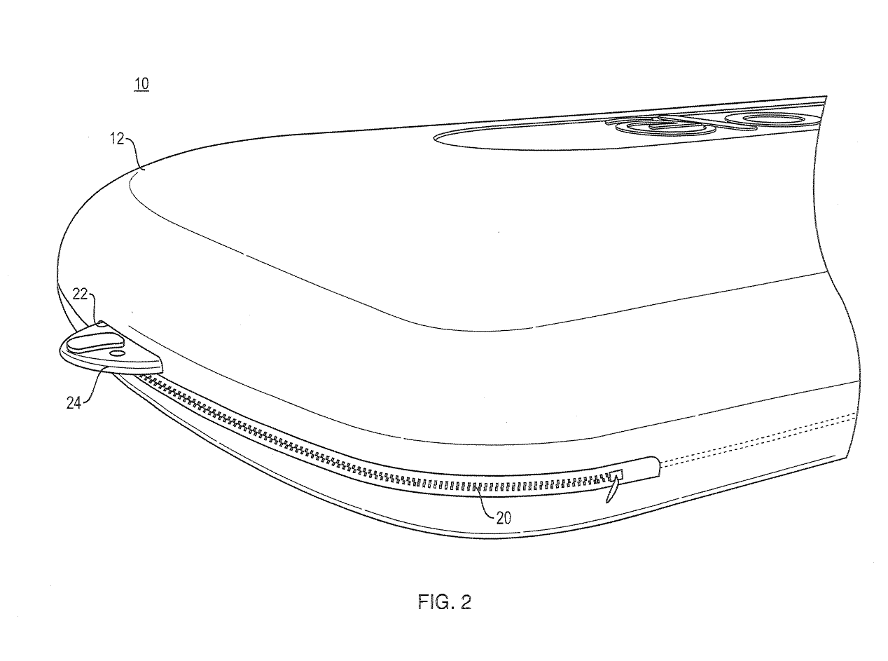

Referring to FIGS. 1-3, a reflex training article of the present invention in the form of a pillow article 10 includes an outer cover 12, an inner cover 14, a fill body 16 and an electronic component 18. The outer cover 12 is formed of a water resistant material such as a plastic material, for example. The pillow 10 is sized and shaped so that it may be covered with a conventional pillow case. The outer cover 12 includes means for opening it, such as a zipper 20, to access an interior thereof. The outer cover 12 may also include an optional first port 22 to allow an access module 24 that may form a part of, or be electrically connected to, the electronic component 18 to extend through the outer cover 12. The outer cover 12 may also include an activation tab, which may be in the form of a tag or tab 30 extending from the pillow body. The electronic component 18 includes memory, which may be separable from a processor of the electronic component and which memory may contain instructions, sound content or a combination of the two. It also includes wireless connectivity.

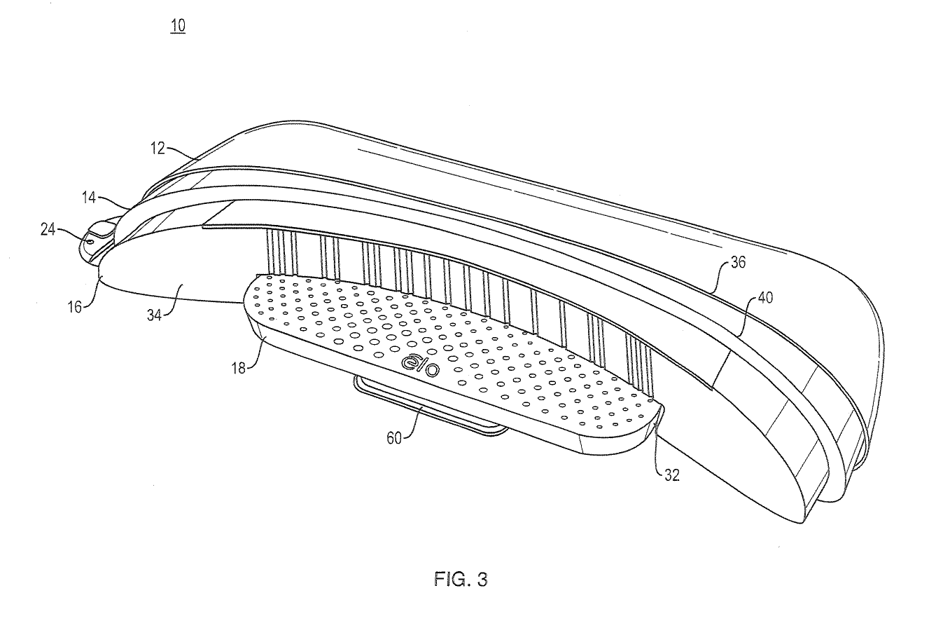

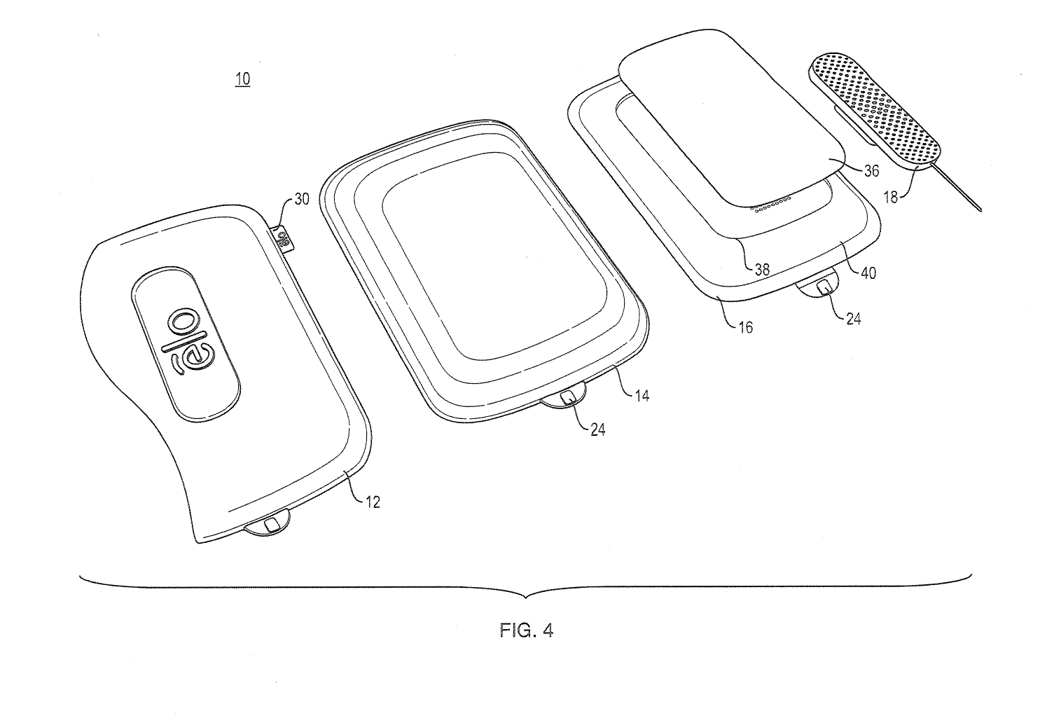

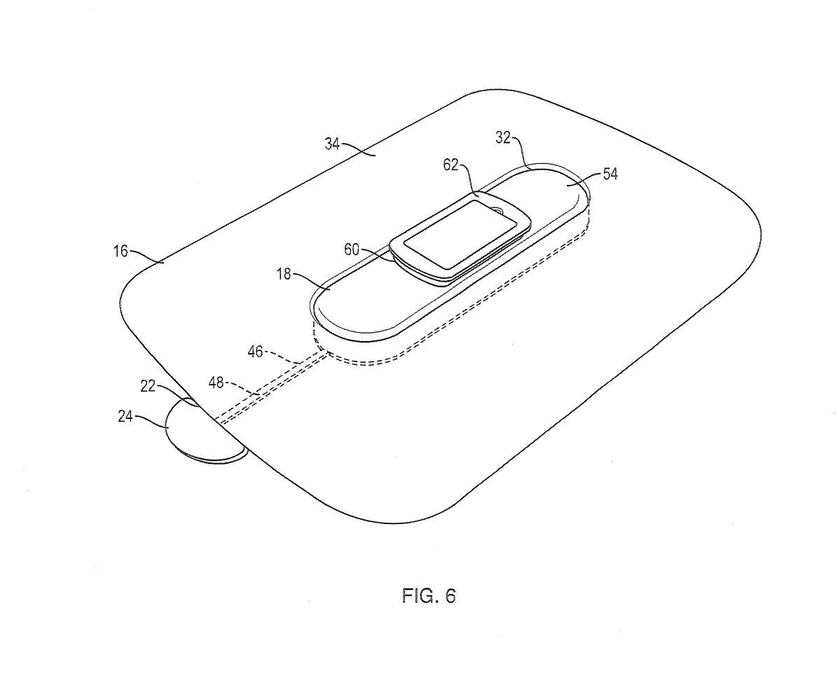

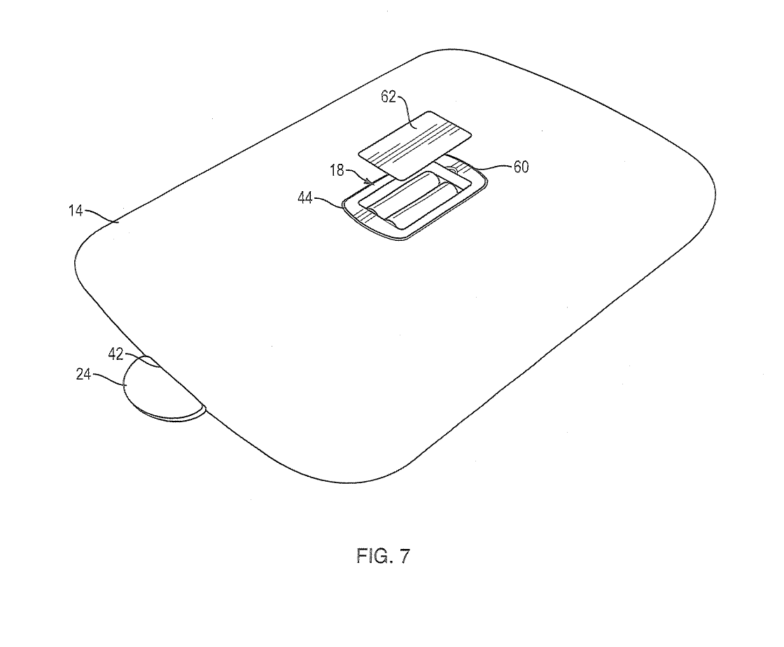

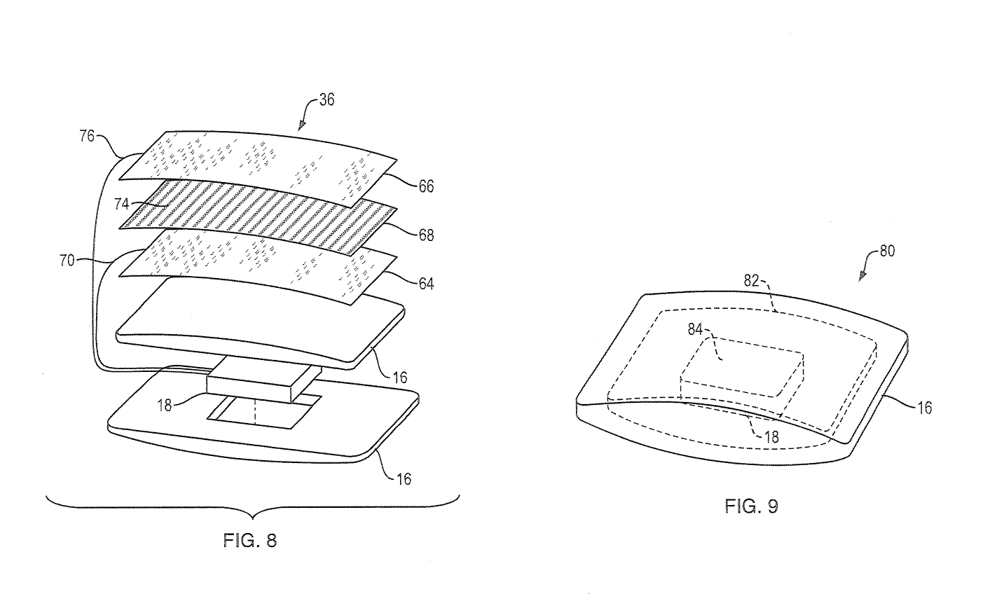

Referring to FIGS. 3-4 and 6-7, the pillow 10 is formed by placing the electronic component 18 in an electronics cavity 32 at an underside 34 of the fill body 16. The fill body 16 may be made of plastic, foam or other suitable material or materials sufficient to retain the electronic component 18 therein and to provide desired physical support to the pillow's user; that is, it may be relatively soft or relatively hard. The fill body 16 may be a unitary structure, as shown in FIG. 3, or it may be formed of two or more pieces placed or joined together, as shown in FIG. 8. A pressure sensor 36 is then placed in a shallow sensor cavity 38 of an upper side 40 of the fill body 16. The fill body 16 also includes an access module conduit 46 extending from the electronics cavity 32 to the first port 22. The conduit 46 is arranged to provide a portal through the fill body 16 for a wiring tube 48 or other type of tubing to allow for electrical connection of the access module 24 to the electronic component 18. The fill body 16 with the electronic component 18 and the pressure sensor 36 therein is then placed in the inner cover 14.

The inner cover 14 may be fabricated of suitable material that may be absorbent, breathable, compliant and otherwise compatible with the fill body 16 and the outer cover 12. The inner cover 14 may be formed of a microfiber fleece material, for example. It may also include an optional first port 42 to allow the access module 24 to extend through the inner cover 14. The inner cover 14 includes an electronic component access opening 44 to allow access to the electronic component 18 retained in the cavity 32 of the fill body 16.

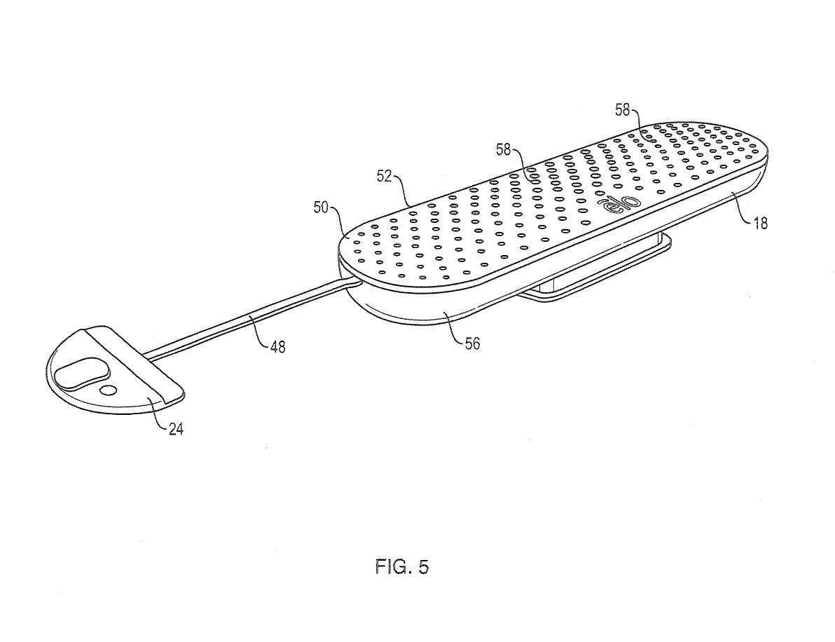

With reference to FIGS. 5-7, the electronic component 18 includes a housing 50 with an upper surface 52, a lower surface 54 and side wall 56. The housing 50 may be made of a rigid plastic material and is sized and shaped to retain the processor, the memory, a sound generator, a transceiver for sending and receiving signals wirelessly and one or more batteries therein. The upper surface 52 may include perforations 58 that allow cooling air to enter the housing 50 to cool the electronics. The lower surface 54 may include a battery well 60 with removable cover 62. The battery well 60 is arranged to retain one or more batteries therein and is accessible through the inner cover 14. The processor may be established in a printed circuit board such as a MCIMX257CJM4A processor running embedded Linux of the type with which electrical engineers in the business of creating and programming circuit boards are familiar. The sound generator may be a Freescale Audio Codec (SGTL5000) coupled with IS31AP4088A Audio amplifier and includes one or more speakers, such as a AS0708C0-R speaker available from Freescale Semiconductors of Austin, Tex. The memory may be a SD Card—Micro SD 512 MB and/or RAM memory card Samsung DDR2 512 MB available from Samsung Austin Semiconductor of Austin, Tex. The transceiver may be a wireless transceiver that is WiFi enabled, such as a JWL-N188R transceiver such as that made available by Shenzhen Yichen (JCG) Technology Development Company of China.

The tag 30 is a tactile membrane switch that is located just outside the pillow body. It is connected to the processor through a cable assembly. The tag 30 has two functions. If it is pressed it will enable the pillow's audible functionality. If the tag 30 is pressed and held for a longer period of time, such as 10 seconds, for example, it will enable wireless connectivity to enable signal exchange with a device or system separate from the pillow 10. The tag 30 or the access module 24 may include a Light Emitting Diode (LED) embedded therein or retained thereto. The LED is an indicator that reflects the functionality that user is triggering by pressing the tag 30.

In general, the pressure sensor 36 is a switch that detects the presence of the user's head resting on the pillow 10. The fill body 16 and/or the inner cover 14 establish a large push button switch area that applies uniform force the pressure sensor 36. The input of the sensor 36 is electrically connected to the power of the electronics component. The output of the pressure sensor 36 is electrically connected to the processor of the electronic component 18, such as through passive filtering, for example, wherein the processor is a printed circuit board, as indicated above. After the pillow 10 is activated by contacting the tag 30, the switch of the pressure sensor 36 is actuated as an indication that the user's head is on the pillow. Other fabric and mechanical versions of the pressure sensor 36 described herein provide. When the pressure sensor 36 is activated, its signal to the processor initiates sound generation in accordance with a selectable program.

The processor is programmed to generate sounds based on instructions provided. The processor is arranged so that the pillow 10 operates in six states. They are: 1) the off state; 2) the power up state; 3) the WiFi state; 4) the play state; 5) the low power state; and 6) the WiFi shutdown state. The circumstances when the state of the pillow changes include: 1) battery installation (power up state); 2) the head leaves the pillow 10 (off state); 3) the head is on the pillow 10 (play state); 4) the tag 30 is depressed (power up state); 5) the tag 30 is pressed and held for an extended period of time (WiFi state); and 6) an idle timeout (WiFi shut down state, low power state, off state). When in the WiFi state, signals may be exchanged such that content may be uploaded to memory.

FIG. 8 shows a first embodiment of a pressure sensor of the present invention as pressure sensor 36. The pressure sensor 36 includes a first conductive fabric layer 64 and a second conductive fabric layer 66 spaced from one another by a compressible insulative layer 68, which may be a compressible foam material, for example. The first and second layers 64 and 66 are formed of or with a conductive material, such as a metal or a conductive plastic material. Each is attached to a respective wire 70, 72, which wires are connected to the electronic component 18. When pressure is applied to the pillow 10, the insulative layer 68 compresses to the point where conductive elements of the first and second layers 64, 66 generate the output signals to the processor as noted above. The insulative layer 68 may have pores 74 arranged so that when the insulative layer 68 is compressed sufficiently, conductive elements of the first and second layers 64, 66 make physical contact to complete the circuit. When the circuit is complete, the processor of the electronic component 18 is activated and sound is generated as previously noted. When the pressure is released, the circuit is open and the processor deactivated. Alternatively, an advisory or instructional message may be generated to change the behavior.

FIG. 9 shows a second embodiment of a pressure sensor of the present invention as pressure sensor 80. The pressure sensor 80 includes a bladder 82 and a pressure switch 84. The bladder may be filled with a compressible fluid such as air. The pressure switch 84 is a mechanical switch that is electrically connected to the processor of the electronic component 18. When pressure is applied to the pillow 10, the pressure of the compressible fluid within the bladder 82 increases to a point that it actuates the pressure switch 84. That actuation activates the processor of the electronic component 18 and sound is generated as previously noted. When the pressure within the bladder 82 is reduced, the pressure switch 84 shuts off and the processor is deactivated. Alternatively, an advisory or instructional message may be generated to change the behavior.

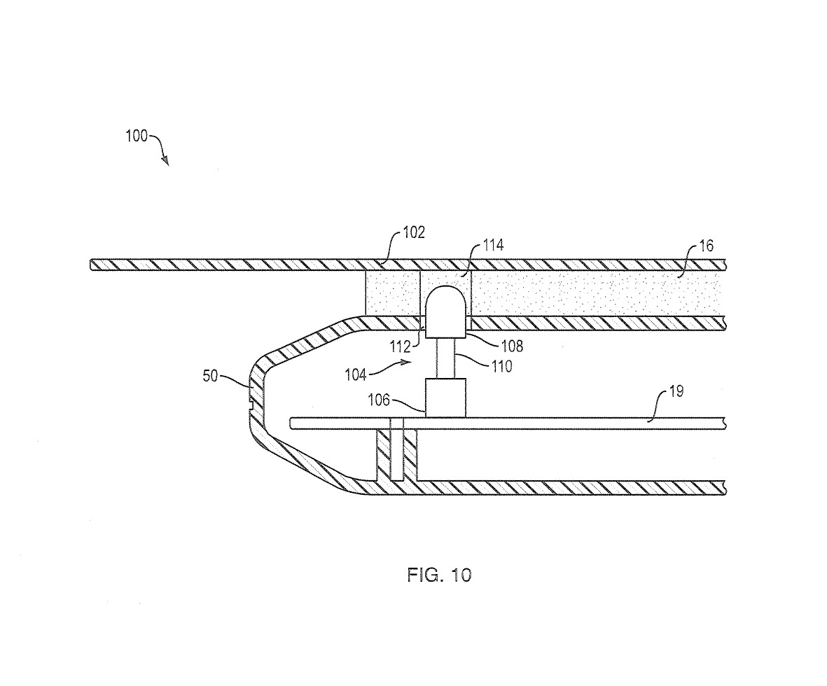

FIG. 10 shows a third embodiment of a pressure sensor of the present invention as pressure sensor 100. The pressure sensor 100 includes a pressure pad 102 and switch 104. The fill body 16 is positioned between the pressure pad 102 and the housing 50. The pressure pad 102 is sufficiently rigid to disperse a substantially uniform load onto the fill body 16 when a head is placed on the pressure pad 102, either directly or indirectly. It may be made of plastic, for example. The fill body 16 has enough give so that when the pressure pad 102 moves toward the housing 50, it will cause displacement of the switch 104. The switch 104 includes a first contact member 106, a second contact member 108 and a spring-loaded body 110. The first contact member 106 is in physical and electrical contact with circuit board 19 of the electronic component 18, which may include the processor. The second contact member 108 is movable on the spring-loaded body 110 such that when sufficient pressure is applied to the pressure pad 102, it forces the second contact member 108 to make contact with the first contact member 106. When that occurs, the processor is activated to cause the generation of sound in accordance with programming. When pressure is removed from the pressure pad 102, the contact of the first contact member 106 and the second contact member 108 is removed and the sound halted. Alternatively, an advisory or instructional message may be generated to change the behavior. In this embodiment of the pillow 10, the housing 50 includes a first spring port 112 that allows the second contact member 108 to be actuated by movement of the pressure pad 102. The fill body 16 may include a second spring port 114 that extends partially or completely through the fill body 16 to enhance the likelihood that minimal pressure applied to the pressure pad 102 is needed to actuate the switch 104.

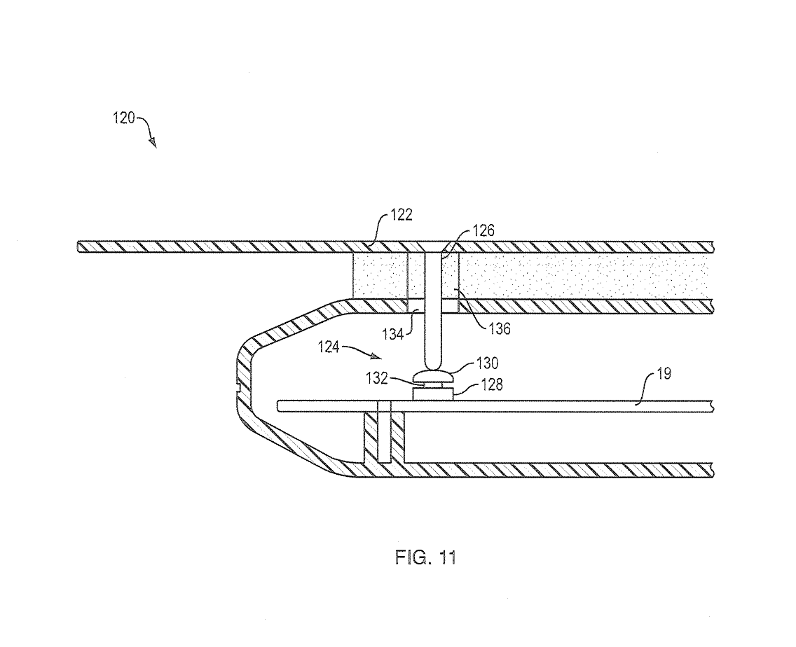

FIG. 11 shows a fourth embodiment of a pressure sensor of the present invention as pressure sensor 120. The pressure sensor 120 includes a pressure pad 122, switch 124 and pressure pin 126. The fill body 16 is positioned between the pressure pad 122 and the housing 50. The pressure pad 122 is sufficiently rigid to disperse a substantially uniform load onto the fill body 16 when a head is placed on the pressure pad 122, either directly or indirectly. It may be made of plastic, for example. The fill body 16 has enough give so that when the pressure pad 122 moves toward the housing 50, it will cause displacement of the switch 124. The switch 124 includes a first contact member 128, a second contact member 130 and a spring-loaded body 132. The first contact member 128 is in physical and electrical contact with circuit board 19 of the electronic component 18, which may include the processor. The second contact member 130 is movable on the spring-loaded body 132 such that when sufficient pressure is applied to the pressure pad 122, it forces the second contact member 130 to make contact with the first contact member 128. The pressure pin 126 is physically connected to an underside surface of the pressure pad 122 and is substantially aligned with the second contact member 130 of the switch 124 such that when the pressure is applied to the pressure pad 122, that pressure is translated to the pressure pin 126. It is arranged to concentrate the load applied by the pressure pad 122 so that it enhances the likelihood that the second contact member 130 will make physical contact with the first contact member 128. When that contact occurs, the processor is activated to cause the generation of sound in accordance with programming. When pressure is removed from the pressure pad 122, the contact of the first contact member 128 and the second contact member 130 is removed and the sound halted. Alternatively, an advisory or instructional message may be generated to change the behavior. In this embodiment of the pillow 10, the housing 50 includes a first pin port 134 that allows the second contact member 130 to be actuated by movement of the pressure pin 126. The fill body 16 may include a second pin port 136 that extends partially or completely through the fill body 16 to enhance the likelihood that minimal pressure applied to the pressure pin 126 is needed to actuate the switch 124.

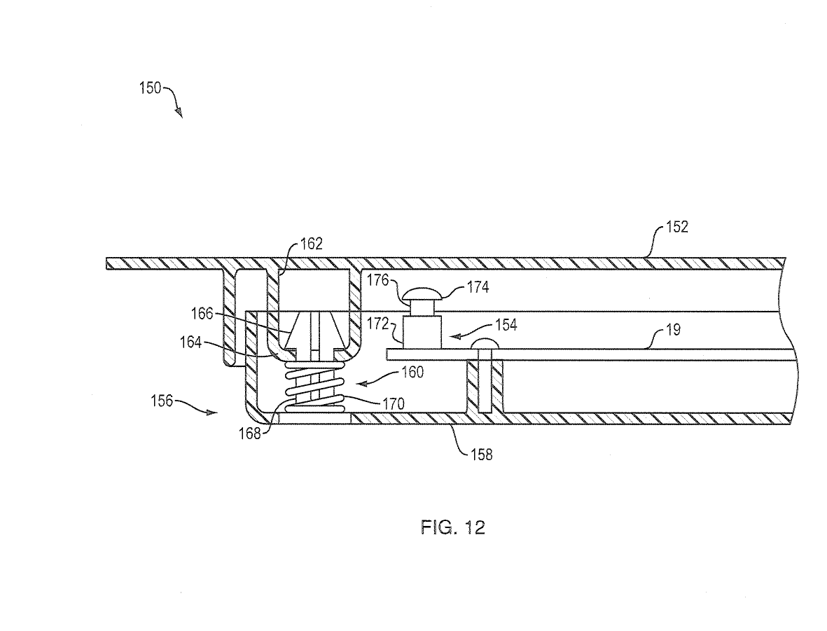

FIG. 12 shows a fifth embodiment of a pressure sensor of the present invention as pressure sensor 150. The pressure sensor 150 includes a pressure pad 152, a switch 154 and modified electronics housing 156. The housing 156 includes a housing body 158 and retained spring boss 160. The pressure sensor 150 does not require fill between the pressure pad 152 and the housing 156. The pressure pad 152 includes a spring boss retainer channel 162. The retainer channel 162 is itself retained to the spring boss 160 with channel well 164 and bolt head 166 of the spring boss 160. The spring boss 160 includes bolt body 168 around which spring 170 extends. The pressure pad 152 is sufficiently rigid to disperse a substantially uniform load onto the spring boss retainer channel 162 when a head is placed on the pressure pad 152, either directly or indirectly. It may be made of plastic, for example. When the pressure pad 152 is moved toward the housing 156, restrained by the force of spring 170, it will make contact with the switch 154 to cause its displacement. The switch 154 includes a first contact member 172, a second contact member 174 and a spring-loaded body 176. The first contact member 172 is in physical and electrical contact with circuit board 19 of the electronic component 18, which may include the processor. The second contact member 174 is movable on the spring-loaded body 176 such that when sufficient pressure is applied to the pressure pad 152 to cause the pressure pad 152 to contact the second contact member 174, it forces the second contact member 174 to make contact with the first contact member 172. When that contact occurs, the processor is activated to cause the generation of sound in accordance with programming. When pressure is removed from the pressure pad 152, the contact of the first contact member 172 and the second contact member 174 is removed and the sound halted or an advisory or instructional message may be generated to change the behavior.

The pillow article 10 is a tool that can be used to optimize the conditions for getting a child to fall to sleep. The method related to the use of the pillow article 10, or another type of article having such sensing and sound or motion generation functionality, may include particular steps to carry out, each of which ends with a sound routine as indicated in Table 1. For purposes of the present invention “elo time” means the transition period when parents leave the nighttime routine and the child develops his or her own sleep skill. The volume, tone and cadence of the particular sleep sounds routine generated by the article 10 or an equivalent of it within a particular age-specific algorithm begins with a recognizable chime, and slowly diminishes in tone, pace and volume until white noise at the close of the routine puts the child to sleep.

TABLE 1

| | | Opening | | Lullabies/ | |
|---|---|---|---|---|---|
| Age | Elo time | chime | Stories | song | Sounds |
| 1-3 | 15-20 mins | 15 secs | 10 mins | 3-5 mins | 2-3 mins |
| 3-6 | 20-30 mins | 5 secs | 15-20 mins | 3-5 mins | 2-3 mins |
| 6-10 | 30-45 | 5 secs | 30-45 | 0 | 2-3 mins |

The present invention provides four primary auditory segments during the elo time. Elo time may begin when the child gets in bed or prior to that. The first is an initial introductory sound that signals to the child that the routine is about to begin. That should start as soon as possible after the child's head or other body part contacts the pillow article 10 or its equivalent. The remaining three segments include three types of audible educational content, which includes a collection of timed audible content found in stories, lullabies and sounds. The routine provides for a mix of these audible elements that begin, after the sound introduction, one or more stories, one or more lullabies and then a tapering off to a library of sounds or white noise. The cadence of sound, stories, songs and diminished tone provide the calming experience required to help a child learn to self sooth and to develop their own positive sleep habits. The sounds may be generated as described herein and may be replaced or supplemented by selectable motions associated with the article 10. While this description of the invention has centered on aiding a child to develop skills effective to enable self control of falling asleep, it may also be useful for someone who is not a child. The generation of sounds and/or movement of a certain type for a certain period of time for each sound type aids the person to get into a routine that optimizes the process of falling asleep. This can be used for other reflex training as well, as earlier noted.

The present invention has been described with respect to a particular embodiment or embodiments. Nevertheless, it is to be understood that various modifications may be made without departing from the spirit and scope of the invention. All equivalents are deemed to fall within the scope of this description of the invention as provided in the following claims.

What is claimed is:

1. An article to train a reflexive condition, the article comprising:
a. a fill body including an electronics component retaining cavity;
b. an electronics component placeable in the retaining cavity of the fill body, wherein the electronics component includes a sound generator configured to generate select sound or sounds;
c. a pressure sensor connected to the electronics component and arranged to activate the sound generator upon sensing a selectable pressure; and
d. a cover for removably retaining the fill body, electronics component and pressure sensor therein.

2. The article of claim 1 further comprising an access module for retaining therein a transceiver for transmitting and receiving electrical signals to and from the electronics component.

3. The article of claim 2 wherein the fill body includes a conduit for retaining therein wiring connecting the transceiver to the electronics component.

4. The article of claim 2 wherein the transceiver is a wireless transceiver.

5. The article of claim 2 wherein the access module includes one or more indicators for indicating the status of the electronics component.

6. The article of claim 1 wherein the pressure sensor includes a first conductive fabric layer and a second conductive fabric layer, wherein the first layer and the second layer are spaced from one another by a compressible insulative layer.

7. The article of claim 6 wherein the compressible insulative layer is a foam material with pores.

8. The article of claim 1 wherein the pressure sensor is a bladder and a pressure switch within the bladder, wherein the bladder includes a compressible fluid therein and is arranged to apply pressure to actuate the pressure switch.

9. The article of claim 1 wherein the electronics component includes a processor arranged to receive and carry out instructions for generating selectable sounds through the sound generator.

10. The article of claim 1 comprising an inner cover and an outer cover.

11. The article of claim 1 wherein the article is a pillow.

12. The article of claim 1 wherein the pressure sensor includes a pressure pad and switch, wherein the switch includes a first contact member, a second contact member and a spring-loaded body, wherein the first contact member is in physical and electrical contact with a circuit board of the electronics component, wherein the second contact member is movable on the spring-loaded body such that when sufficient pressure is applied to the pressure pad, it forces the second contact member to make contact with the first contact member to cause the generation of sound.

13. The article of claim 12 wherein the pressure sensor further includes a pressure pin, wherein the pressure pin is physically connected to an underside surface of the pressure pad and is substantially aligned with the second contact member of the switch such that when the pressure is applied to the pressure pad, that pressure is translated to the pressure pin.

14. The article of claim 1 wherein the pressure sensor includes a pressure pad, a switch and modified electronics housing, wherein the modified housing includes a housing body and retained spring boss, wherein the pressure pad includes a spring boss retainer channel that is retained to the spring boss with a channel well and bolt head of the spring boss, wherein the spring boss includes a bolt body around which a spring extends, wherein the pressure pad is sufficiently rigid to disperse a substantially uniform load onto the spring boss retainer channel when pressure is applied thereto, and wherein when the pressure pad is moved toward the housing it will make contact with the switch to cause its displacement and activate a processor to cause the generation of sound in accordance with programming.
